(19) United States
(12) Patent Application Publication
(10) Pub. No.: US 2026/0102598 A1
(43) Pub. Date: Apr. 16, 2026

(54) PASSIVE TUOHY COMBINATION CATHETER SEAL

(71) Applicant: BOSTON SCIENTIFIC SCIMED, INC., MAPLE GROVE, MN (US)

(72) Inventor: Ryan Robert Davis, Plymouth, MN (US)

(73) Assignee: BOSTON SCIENTIFIC SCIMED, INC., MAPLE GROVE, MN (US)

(21) Appl. No.: 19/359,126

(22) Filed: Oct. 15, 2025

Related U.S. Application Data
(60) Provisional application No. 63/707,334, filed on Oct. 15, 2024.

(51) Int. Cl. A61M 39/06

(52) U.S. Cl. CPC A61M 39/0613; A61M 2039/062; A61M 2039/0633

(57) ABSTRACT

An access sheath valve device includes an elongate body defining an inner chamber, a valve seal disposed within the inner chamber, a lip seal member fixed to an inner circumferential surface of the valve seal, and a compression member disposed on the elongate body and configured to be moved between a first position allowing the valve seal to be in an opened configuration and a second position which compresses the valve seal into a closed configuration. The lip seal member is configured to passively seal around a catheter disposed through the valve seal while the compression member is in the first position and the valve seal is in the opened configuration.

### CROSS-REFERENCE TO RELATED APPLICATIONS

This application claims the benefit of priority of U.S. Provisional Application No. 63/707,334 filed October 15, 2024, the entire disclosure of which is hereby incorporated by reference.

### TECHNICAL FIELD

The disclosure relates generally to medical devices and more particularly to guide catheters including access sheaths that provide a fluid-tight access through the guide catheter hub for other medical devices.

### BACKGROUND

A wide variety of medical devices have been developed for medical use including, for example, intravascular procedures. Access sheaths and sealing mechanisms play a crucial role in various medical procedures, particularly in cardiovascular interventions. These devices facilitate the introduction and manipulation of catheters and other instruments while maintaining hemostasis. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example access sheath valve device includes an elongate body defining an inner chamber, a valve seal disposed within the inner chamber, the valve seal having an inner circumferential surface, a lip seal member fixed to the inner circumferential surface of the valve seal, and a compression member disposed on the elongate body and configured to be moved between a first position allowing the valve seal to be in an opened configuration and a second position which compresses the valve seal into a closed configuration. T he lip seal member may be configured to passively seal around a catheter disposed through the valve seal while the compression member is in the first position and the valve seal is in the opened configuration.

Alternatively or additionally to the embodiment above, the lip seal member extends radially inward from the inner circumferential surface of the valve seal.

Alternatively or additionally to any of the embodiments above, the lip seal member extends 0.25 mm to 1.5 mm radially inward of the inner circumferential surface of the valve seal.

Alternatively or additionally to any of the embodiments above, the lip seal member is made of silicone.

Alternatively or additionally to any of the embodiments above, the lip seal member is configured to passively seal against catheters with a diameter range of 5-13 Fr while the valve seal is in the opened configuration.

Alternatively or additionally to any of the embodiments above, the lip seal member extends perpendicular to the inner circumferential surface of the valve seal.

Alternatively or additionally to any of the embodiments above,

the lip seal member extends radially inward and leans toward a distal end of the elongate body, creating a funnel effect.

Alternatively or additionally to any of the embodiments above, the lip seal member has a first end fixed to a midpoint on the inner circumferential surface and a second free end extending to a distal end of the inner circumferential surface of the valve seal.

Alternatively or additionally to any of the embodiments above, the compression member is a threaded cap threadingly engaged with the elongate body, wherein the first position is a loosened position and the second position is a tightened position.

Alternatively or additionally to any of the embodiments above, the threaded cap includes an outer portion configured to threadingly engage outer threading on the elongate body, and an inner portion configured to be disposed within the inner chamber adjacent the valve seal in the first position, wherein moving the threaded cap from the first position to the second position moves the inner portion into contact with the valve seal, compressing the valve seal.

An example system for sealing an access sheath includes an access sheath housing, a valve seal disposed within the access sheath housing, the valve seal having an inner circumferential surface defining an axial opening configured to receive a catheter shaft, a lip seal member fixed to the inner circumferential surface of the valve seal and a compression member disposed on the access sheath housing, the compression member including an outer portion disposed on an outer surface of the access sheath housing and an inner portion disposed within an inner chamber of the access sheath housing, the compression member configured to be moved between a first position in which the valve seal is in an opened configuration and a second position in which the inner portion compresses the valve seal into a closed configuration. The lip seal member may be configured to passively seal against a catheter extending through the valve seal while the valve seal is in the opened configuration.

Alternatively or additionally to any of the embodiments above, the lip seal member extends radially inward from the inner circumferential surface of the valve seal.

Alternatively or additionally to any of the embodiments above, the lip seal member extends 0.25 mm to 1.5 mm radially inward of the inner circumferential surface of the valve seal.

Alternatively or additionally to any of the embodiments above,

the lip seal member extends perpendicular to the inner circumferential surface of the valve seal.

Alternatively or additionally to any of the embodiments above, the lip seal member is configured to form a passive seal on catheters with a diameter range of 5-13 Fr.

Alternatively or additionally to any of the embodiments above, the lip seal member extends radially inward and leans toward a distal end of the access sheath housing, creating a funnel effect.

Alternatively or additionally to any of the embodiments above, the lip seal member has a first end fixed to a midpoint on the inner circumferential surface and a second free end extending to a distal end of the inner circumferential surface of the valve seal.

Alternatively or additionally to any of the embodiments above, the compression member is a threaded cap threadingly engaged with the access sheath housing, wherein the first position is a loosened position and the second position is a tightened position.

Alternatively or additionally to any of the embodiments above, the outer portion of the threaded cap is configured to threadingly engage outer threading on the access sheath housing, and the inner portion is configured to be disposed within the inner chamber adjacent the valve seal in the first position, wherein moving the threaded cap from the first position to the second position moves the inner portion into contact with the valve seal, compressing the valve seal.

Another example access sheath valve device may include a housing defining an inner chamber, a valve seal disposed within the inner chamber, the valve seal having an inner circumferential surface defining an axial opening configured to receive a catheter shaft, the valve seal moveable between a first, open configuration, and a second, closed configuration, wherein an inner diameter of the axial opening is smaller in the second, closed configuration than in the first, open configuration, a lip seal member extending radially inward from the inner circumferential surface of the valve seal, and a compression member disposed on the housing and configured to be moved between a first position in which the valve seal is in the open configuration, and a second position in which a portion of the compression member directly contacts the valve seal and compresses the valve seal into the closed configuration. The lip seal member may be configured to passively seal around a catheter disposed through the axial opening in the valve seal while the compression member is in the first position and the valve seal is in the open configuration.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A is a longitudinal cross-sectional view of an access sheath with a valve seal disposed therein and a compression member poised for insertion;

FIG. 1B is a longitudinal cross-sectional view of the access sheath of FIG. 1A with the compression member inserted;

FIG. 2 is a perspective view of the valve seal of FIG. 1;

FIG. 3 is a cross-sectional view of the valve seal taken through line 3-3 of FIG. 2;

FIG. 4 is a perspective view of another valve seal; and

FIG. 5 is a cross-sectional view of the valve seal taken through line 5-5 of FIG. 4.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “ and/or ” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “ proximal ”, “ distal ”, “advance”, “withdraw”, variants thereof, and the like, may be generally considered with respect to the positioning, direction, and/or operation of various elements relative to a user/operator/manipulator of the device, wherein “ proximal ” and “withdraw” indicate or refer to closer to or toward the user and “ distal ” and “advance” indicate or refer to farther from or away from the user. In some instances, the terms “ proximal ” and “ distal ” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “ outer extent ” may be understood to mean a maximum outer dimension, “ radial extent ” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “ minimum extent ” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “ an embodiment ”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “ second ” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified .

The conventional access sheath for a pulmonary vein isolation (PVI) procedure followed concomitantly by a left atrial appendage closure (LAAC) procedure includes a Tuohy seal system at the proximal end to achieve hemostasis and reduce blood loss during the procedure. These procedures often involve the use of catheters and delivery sheaths of varying sizes, necessitating effective sealing to prevent blood loss while maintaining ease of instrument manipulation. Tuohy valve seals commonly feature an adjustable mechanism that can be tightened or loosened to accommodate different instrument sizes. The Tuohy seal is actively open until it is manually closed, generally by rotating an outer compression member. The Tuohy seal has a smooth inner surface. The PVI catheter outer diameter (OD) is typically smaller than a 12 French (Fr) delivery sheath commonly used to deliver an LAAC device. The use of a Tuohy style seal where it is fully open until manually closed may allow for blood fluid to pass by the PVI catheter-to-Tuohy Seal interface during a procedure, resulting in unintended blood loss . This has been witnessed via benchtop studies with in-vivo relevant (~120 mmHg) pressurized benchtop anatomical models with access sheaths with standard, iso-diametric Tuohy valve seals and PVI catheters. It is often difficult to tighten down the current Tuohy valve such that it adequately prevents unintended blood loss while simultaneously allowing for the user to slide the PVI catheter at their discretion while it is inserted across the Tuohy valve seal, and some users cannot achieve this ideal state. This may result in user dissatisfaction and desire for an access sheath seal that does not require manual tightening during use with the PVI catheter.

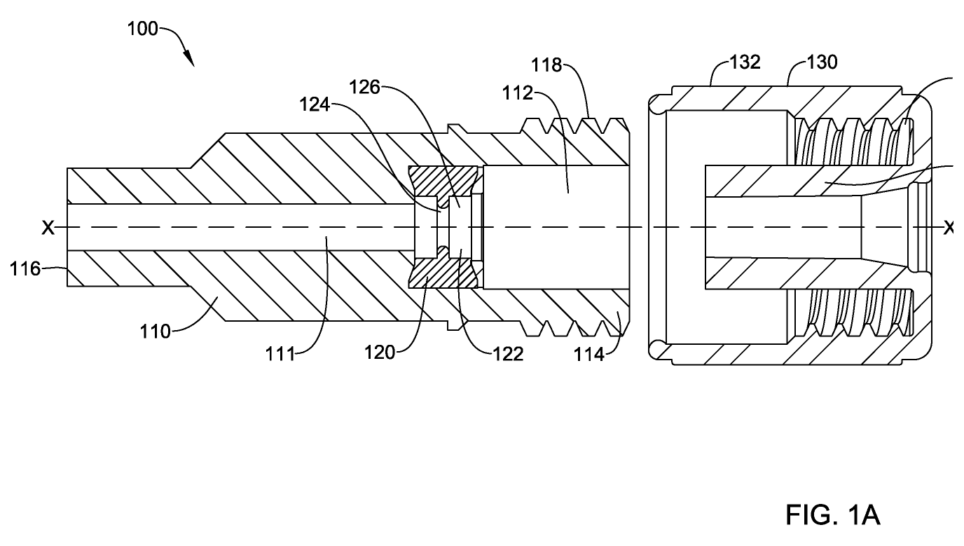

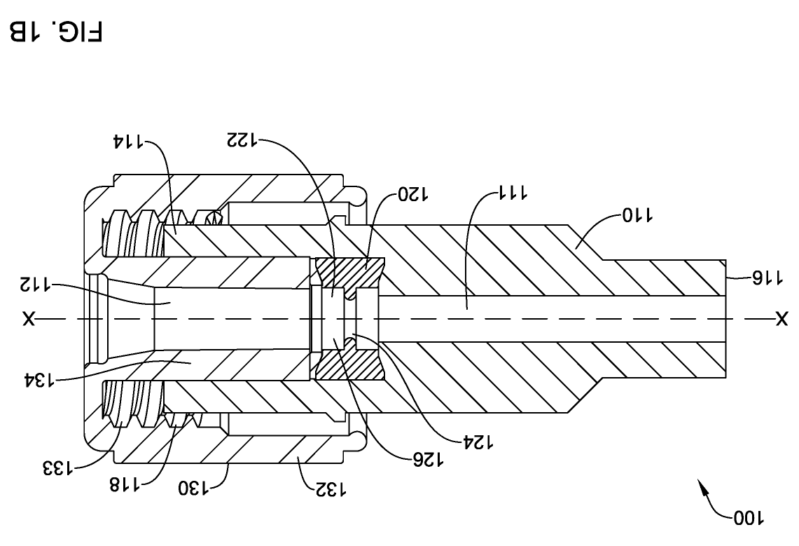

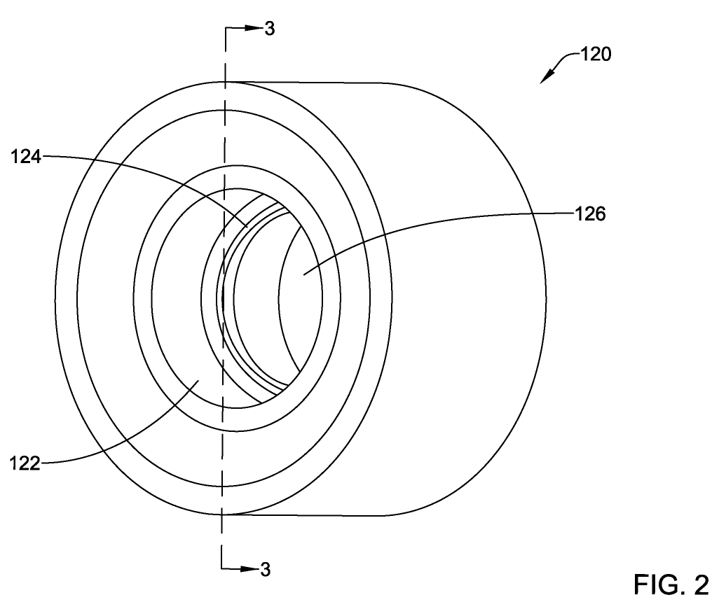

FIG. 1A illustrates an access sheath valve device 100 that provides effective sealing during medical procedures utilizing small diameter devices while the valve is open. The access sheath valve device 100 may include an elongate body 110 extending from a proximal end 114 to a distal end 116 along a longitudinal axis X-X. The elongate body 110 may be a housing or an access sheath housing. The elongate body 110 may define a lumen 111 extending through a distal section and an inner chamber 112 defined in a proximal section. The combination of the lumen 111 and the inner chamber 112 may extend from the proximal end 114 to the distal end 116 of the elongate body 110, and may be configured to receive a medical device such as a catheter. The distal end 116 may be configured for attachment to a hub. A valve seal 120 may be disposed within the inner chamber 112 at the junction with the lumen 111. The valve seal 120 may generally be cylindrical in shape with an axial opening 126 extending axially through a center of the valve seal 120, as shown in FIG. 2. The axial opening 126 defines a lumen which may align with the lumen 111 and inner chamber 112, as shown in FIG. 1A. The valve seal 120 may have an inner circumferential surface 122 and a lip seal member 124 fixed to the inner circumferential surface 122. The remainder of the inner circumferential surface 122, with the exception of the lip seal member 124, may be smooth. In various embodiments, the valve seal 120 may be made of silicone, or various other suitable materials like polymer, thermoset, rubber, or thermoset elastomer (TSE). Various embodiments of the valve seal 120 will be described further herein with reference to FIGS. 2-5.

The proximal end 114 of the elongate body 110 may have external threads 118 configured to threadingly engage a compression member 130. In some embodiments, the compression member 130 may include an outer portion 132 having an inner surface that defines a threaded portion 133 which is configured for engagement with the external threads 118 on the elongate body 110. The compression member 130 may additionally include an inner portion 134 configured for extending within the proximal end of the inner chamber 112. The compression member 130 may be disposed on the elongate body 110, with the outer portion 132 threadingly engaged with the external threads 118 on the elongate body and the inner portion 134 inserted into the proximal end of the inner chamber 112 adjacent the valve seal 120, as shown in FIG. 1B. The compression member 130 may thus be a threaded cap threadingly engaged with the elongate body 110 and configured to be moved between a first, loosened position allowing the valve seal 120 to be in an opened configuration and a second, tightened position which compresses the valve seal 120 into a closed configuration. The inner diameter of the axial opening 126 of the valve seal 120 is smaller in the closed configuration as compared to in the open configuration. With the compression member 130 positioned over the proximal end 114 of the elongate body 110, the compression member 130 may be rotated by the operator in order to tighten the compression member 130 onto the elongate body 110. As the compression member 130 is tightened onto the elongate body 110, the inner portion 134 moves distally into direct contact with the valve seal 120 and then compresses the valve seal 120 as the inner portion 134 moves axially along the longitudinal axis X-X through the inner chamber 112.

As illustrated in FIG. 1A, the valve seal 120 is positioned at a transition point of the elongate body 110, wherein the lumen 111 transitions into the inner chamber 112 and the diameter increases from a first diameter in the lumen 111 to a second diameter in the inner chamber 112 that is larger than the first diameter. As previously mentioned, during engagement of the compression member 130 and the elongate body 110, the inner portion 134 is positioned within the inner chamber 112 while the outer portion 132 is positioned around the outer surface of the elongate body110. FIG. 1B is a longitudinal cross-sectional view of the access sheath of FIG. 1A with the compression member inserted. As illustrated in FIG. 1B, once the compression member 130 is tightened into engagement with the elongate body 110, the inner portion 134 may be in direct contact with the valve seal 120. During engagement and displacement of the inner portion 134 further distally into the inner chamber 112, the inner portion 134 axially compresses the valve seal 120. Due to the positioning of the valve seal 120 near the transition point between the lumen 111 and the inner chamber 112 of the elongate body 110, the axial compression of the valve seal 120 reduces the diameter of the axial opening 126, thereby actively sealing around a catheter shaft or other device inserted through the valve seal 120. The axial compression also causes the valve seal 120 to expand, for example radially and/or axially, against an inner surface of the elongate body 110 within the inner chamber 112. In this way, the valve seal 120 forms a fluid seal, preventing fluids such as blood, saline, drugs, or anything injected into the system from leaking out proximally through the valve seal 120 into the inner chamber 112. Axial compression of the valve seal 120 also assures that any catheter or device inserted through the valve seal 120 is secured and stabilized, such that radial or axial movement of the catheter or other medical device is substantially reduced or eliminated.

Some procedures, such as pulmonary vein isolation (PVI) performed with left atrial appendage closure (LAAC) require catheter or delivery shafts of differing diameters to be inserted in rapid succession through the valve seal 120, and it may be cumbersome and/or difficult to tighten down the compression member 130 around the thinner catheter shaft to avoid blood loss while also inserting a larger catheter shaft. The valve seal 120 provides a solution in the presence of the lip seal member 124 fixed to the inner circumferential surface 122 of the valve seal 120. The lip seal member 124 may be a passive seal, configured to passively seal around a smaller diameter catheter disposed through the valve seal 120 while the compression member 130 is in the first position and the valve seal 120 is in the opened configuration. The lip seal member 124 may prevent blood and any other fluid loss around smaller diameter catheters while minimizing resistance during insertion of larger delivery sheaths.

The lip seal member 124 may be formed with the valve seal 120 as a single, monolithic piece that extends around the entire circumference of the inner surface of the valve seal 120. In other embodiments, the lip seal member 124 may be formed separately and fixed to the inner circumferential surface 122 of the valve seal 120. The lip seal member 124 may extend radially inward from the inner circumferential surface 122 of the valve seal 120. The inner diameter of the valve seal 120 in the region of the inner circumferential surface 122 may be 4.0 mm to 5.0 mm. In one embodiment, the inner diameter of the inner circumferential surface 122 may be 4.5 mm. In some embodiments, the lip seal member may extend 0.25 mm to 1.5 mm radially inward of the inner circumferential surface 122 of the valve seal. The lip seal member 124 may be configured to passively seal against catheters with an outer diameter range of 5-13 Fr (1.67 mm to 4.33 mm) while the valve seal 120 is in the open configuration.

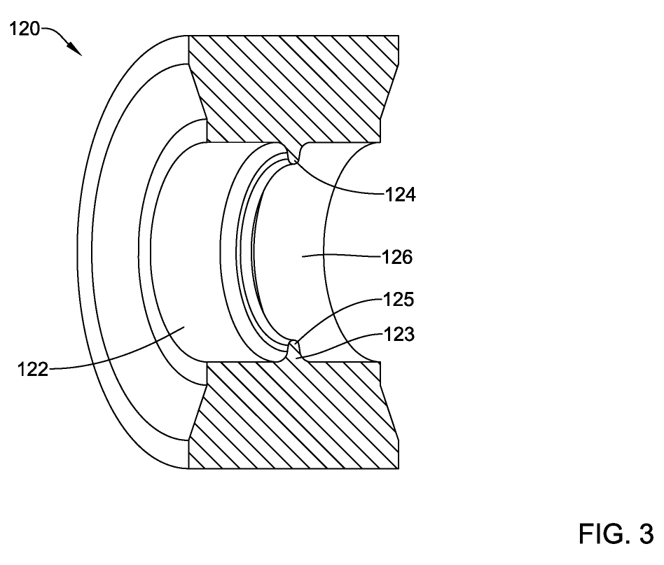

FIG. 2 is a perspective view of the valve seal of FIG. 1 and FIG. 3 is a cross-sectional view of the valve seal taken through line 3-3 of FIG. 2. In the embodiment shown in FIGS. 2-3, the lip seal member 124 is positioned centrally along the inner circumferential surface 122, as measured in the axial direction. In other embodiments, the lip seal member 124 may be positioned closer to the distal or proximal end of the valve seal 120, at any axial position along the inner circumferential surface 122. The lip seal member 124 as shown extends radially inward and perpendicular to the inner circumferential surface 122 of the valve seal 120. The perpendicular orientation and central position makes the valve seal 120 symmetrical and allows for the valve seal 120 to be inserted into the elongate body 110 in either direction. The lip seal member 124 may have a flared first end 123 attached to the inner circumferential surface 122, and a rounded second free end 125.

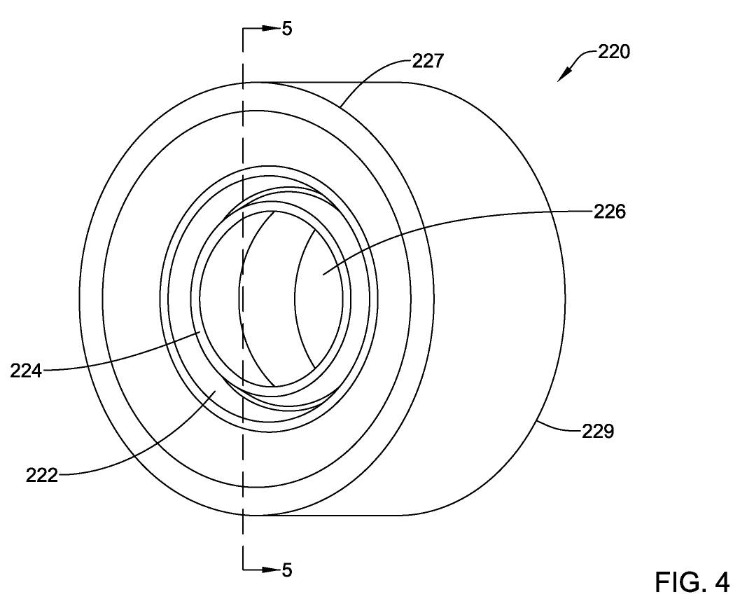

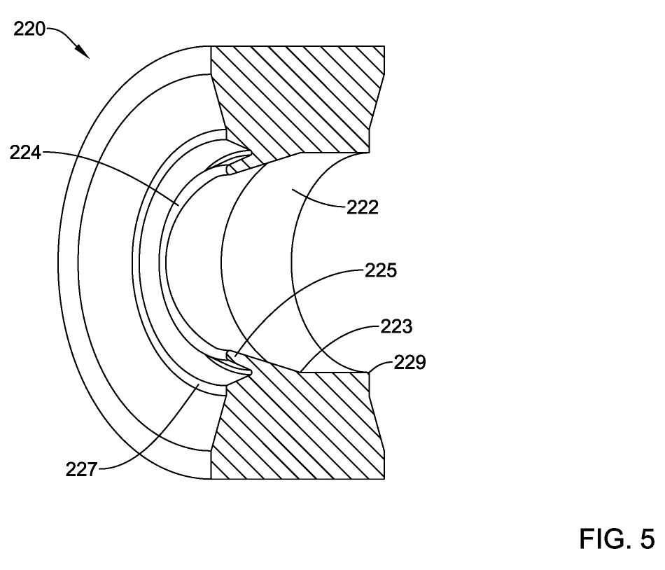

FIG. 4 is a perspective view of another valve seal and FIG. 5 is a cross-sectional view of the valve seal taken through line 5-5 of FIG. 4. FIGS. 4 and 5 show another embodiment of a valve seal 220 configured to be inserted into the elongate body 110 shown in FIG. 1 and described above. The valve seal 220 is similar to valve seal 120, with a different configuration of lip seal member 224. In this embodiment, the lip seal member 224 has an elongate shape that extends radially inward from the inner circumferential surface 222 but instead of being perpendicular, the lip seal member 224 leans toward the distal end 227 of the inner circumferential surface 222 of the valve seal 220. The lip seal member 224 may have a first end 223 fixed to a midpoint on the inner circumferential surface 222, midway between the distal end 227 and the proximal end 229 of the inner circumferential surface 222. The lip seal member 224 extends from the first end 223 to a second, free end 225. In other embodiments, the first end 223 of the lip seal member 224 may extend from the inner circumferential surface 222 at a position closer to the proximal end 229 or the distal end 227. In some embodiments, the lip seal member 224 may extend at an angle of between 10 degrees and 45 degrees relative to the inner circumferential surface 222. The second, free end 225 may extend partially or all the way to the distal end 227 of the inner circumferential surface 222 of the valve seal 220. In other embodiments, the second, free end 225 of the lip seal member 224 may extend distally beyond the distal end 227 of the inner circumferential surface 222. In some embodiments, the elongate lip seal member 224 may have a lateral thickness of between 0.25 mm and 1.5 mm and a length of between 0.5 mm and 4 mm measured between the first end and the second, free end 225. The second, free end 225 of the elongate lip seal member 224 has a circumference smaller than the circumference of the inner circumferential surface 222, such that the lip seal member 224 creates a funnel effect that minimizes loading on a catheter tip inserted through the valve seal 220, and facilitates easier insertion of both catheters and delivery sheaths. This design allows for successful left atrial appendage closure implant deployment while maintaining effective sealing during PVI procedures.

Both lip seal member designs discussed above offer several advantages over traditional Tuohy valve seals alone. They provide passive sealing on smaller diameter catheters without requiring manual adjustment, reducing the risk of unintended blood loss during procedures.

The designs also minimize resistance during the insertion of larger delivery sheaths, facilitating smoother transitions between different instruments during complex cardiovascular interventions.

It will be understood that the dimensions described in association with the above figures are illustrative only, and that other dimensions of valve seals 120, 220 and lip seal members 124, 224 are contemplated. The materials that can be used for the various components of the access sheath valve device 100 (and/or other systems or components disclosed herein) and the various elements thereof disclosed herein may include those commonly associated with medical devices.

In some embodiments, the access sheath valve device 100 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY ® C-22® , UNS: N10276 such as HASTELLOY ® C276®, other HASTELLOY® alloys, and the like), nickel-copper alloys (e.g., UNS: N04400 such as MONEL® 400, NICKELVAC ® 400, NICORROS ® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated "linear elastic" or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial "super-elastic plateau" or "flag region" in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about –60 degrees Celsius (ºC) to about 120 ºC in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In some embodiments, the access sheath valve device 100 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene- b -isobutylene- b -styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An access sheath valve device, comprising:
an elongate body defining an inner chamber;
a valve seal disposed within the inner chamber, the valve seal having an inner circumferential surface;
a lip seal member fixed to the inner circumferential surface of the valve seal; and
a compression member disposed on the elongate body and configured to be moved between a first position allowing the valve seal to be in an opened configuration and a second position which compresses the valve seal into a closed configuration;
wherein the lip seal member is configured to passively seal around a catheter disposed through the valve seal while the compression member is in the first position and the valve seal is in the opened configuration.

2. The access sheath valve device of claim 1, wherein the lip seal member extends radially inward from the inner circumferential surface of the valve seal.

3. The access sheath valve device of claim 1, wherein the lip seal member extends 0.25 mm to 1.5 mm radially inward of the inner circumferential surface of the valve seal.

4. The access sheath valve device of claim 1, wherein the lip seal member is made of silicone.

5. The access sheath valve device of claim 1, wherein the lip seal member is configured to passively seal against catheters with a diameter range of 10-12 Fr while the valve seal is in the opened configuration.

6. The access sheath valve device of claim 1, wherein the lip seal member extends perpendicular to the inner circumferential surface of the valve seal.

7. The access sheath valve device of claim 1, wherein the lip seal member extends radially inward and leans toward a distal end of the elongate body, creating a funnel effect.

8. The access sheath valve device of claim 7, wherein the lip seal member has a first end fixed to a midpoint on the inner circumferential surface and a second free end extending to a distal end of the inner circumferential surface of the valve seal.

9. The access sheath valve device of claim 1, wherein the compression member is a threaded cap threadingly engaged with the elongate body, wherein the first position is a loosened position and the second position is a tightened position.

10. The access sheath valve device of claim 9, wherein the threaded cap includes an outer portion configured to threadingly engage outer threading on the elongate body, and an inner portion configured to be disposed within the inner chamber adjacent the valve seal in the first position, wherein moving the threaded cap from the first position to the second position moves the inner portion into contact with the valve seal, compressing the valve seal.

11. A system for sealing an access sheath, comprising:
an access sheath housing;
a valve seal disposed within the access sheath housing, the valve seal having an inner circumferential surface defining an axial opening configured to receive a catheter shaft;
a lip seal member fixed to the inner circumferential surface of the valve seal; and
a compression member disposed on the access sheath housing, the compression member including an outer portion disposed on an outer surface of the access sheath housing and an inner portion disposed within an inner chamber of the access sheath housing, the compression member configured to be moved between a first position in which the valve seal is in an opened configuration and a second position in which the inner portion compresses the valve seal into a closed configuration;
wherein the lip seal member is configured to passively seal against a catheter extending through the valve seal while the valve seal is in the opened configuration.

12. The system of claim 11, wherein the lip seal member extends radially inward from the inner circumferential surface of the valve seal.

13. The system of claim 11, wherein the lip seal member extends 0.25 mm to 1.5 mm radially inward of the inner circumferential surface of the valve seal.

14. The system of claim 11, wherein the lip seal member extends perpendicular to the inner circumferential surface of the valve seal.

15. The system of claim 11, wherein the lip seal member is configured to form a passive seal on catheters with a diameter range of 5-13 Fr.

16. The system of claim 16, wherein the lip seal member extends radially inward and leans toward a distal end of the access sheath housing, creating a funnel effect.

17. The system of claim 11, wherein the lip seal member has a first end fixed to a midpoint on the inner circumferential surface and a second free end extending to a distal end of the inner circumferential surface of the valve seal.

18. The system of claim 11, wherein the compression member is a threaded cap threadingly engaged with the access sheath housing, wherein the first position is a loosened position and the second position is a tightened position.

19. The system of claim 18, wherein the outer portion of the threaded cap is configured to threadingly engage outer threading on the access sheath housing, and the inner portion is configured to be disposed within the inner chamber adjacent the valve seal in the first position, wherein moving the threaded cap from the first position to the second position moves the inner portion into contact with the valve seal, compressing the valve seal.

20. An access sheath valve device, comprising:
a housing defining an inner chamber;
a valve seal disposed within the inner chamber, the valve seal having an inner circumferential surface defining an axial opening configured to receive a catheter shaft, the valve seal moveable between a first, open configuration, and a second, closed configuration, wherein an inner diameter of the axial opening is smaller in the second, closed configuration than in the first, open configuration;
a lip seal member extending radially inward from the inner circumferential surface of the valve seal; and
a compression member disposed on the housing and configured to be moved between a first position in which the valve seal is in the open configuration, and a second position in which a portion of the compression member directly contacts the valve seal and compresses the valve seal into the closed configuration;
wherein the lip seal member is configured to passively seal around a catheter disposed through the axial opening in the valve seal while the compression member is in the first position and the valve seal is in the open configuration.
